(19) United States
(12) Patent Application Publication
(10) Pub. No.: US 2021/0223278 A1
(43) Pub. Date: Jul. 22, 2021

(54) Reusable Estrus Detection System

(71) Applicant: ST Reproductive Technologies, LLC, Navasota, TX (US)

(72) Inventors: Nicholas P. Rettedal, Berthoud, CO (US); Stephen M. Weilnau, Greeley, CO (US)

(73) Assignee: ST Reproductive Technologies, LLC, Navasota, TX (US)

(21) Appl. No.: 17/152,410

(22) Filed: Jan. 19, 2021

Related U.S. Application Data
(60) Provisional application No. 62/963,620, filed on Jan. 21, 2020.

(51) Int. Cl. G01P 1/02; A61D 17/00

(52) U.S. Cl. CPC G01P 1/023; A61D 17/002

(57) ABSTRACT

A reusable estrus detection system includes disposable components and reusable components that interlock during operation. Following use, the disposable components can be disengaged and replaced by interlocking a new set of disposable components with the reusable components.

### REFERENCE TO RELATED APPLICATIONS

This application claims the benefit of U.S. Provisional Patent Application No. 62/963,620 filed Jan. 21, 2020. The entire disclosure of which is incorporated herein by reference.

### BACKGROUND OF INVENTION

In the livestock industry reproductive outcomes significantly impact a producer's profitability. Negative reproductive outcomes in a breeding program impose both actual costs, such as the cost tied up in semen and veterinary technicians, and opportunity costs, such as the unavailability of inseminated animals. Numerous practices and products developed over the years aim to improve these reproductive outcomes. One particular practice focuses on identifying animal behavior corresponding to the animal's ovulation cycle. Correlating certain animal behavior to its ovulation cycle allows the producer to perform artificial insemination in a window with higher conception rates and thus better reproductive outcomes.

Mounting behaviors present one such reliable correlation between an observable, quantifiable activity and an animal's ovulation cycle. In particular, monitoring the timing and frequency of mounting attempts reliably permits the determination of heat or estrus in bovids. Based on the timing and frequency of mounting attempts artificial insemination can then be performed in a specified time frame following thereafter to improve the likelihood of conception. Ideally, artificial insemination occurs a specified number of hours after a peak frequency of mounting attempts. Producers, however, cannot always be present to monitor and quantify such behaviors. A significant limitation for this practice, therefore, resides in its reliance on observation.

Several developments in the field allow producers to make such problematic observations less frequently and still obtain a general idea of when the animal ovulated. However, much room for improvement remains. Products in the form of electronic devices are available with appropriate sensors and communication means for pinpointing the exact timing of a mounting event and relaying that information to the producer in near real time. Such devices require numerous electrical components to facilitate the storage, processing, and transmission of relevant data. Simply attaching such an device with all its necessary electrical components may be cost prohibitive, as the prior devices are suitable for a single use.

In particular, the limitations of the adhesives used and the significant pressure and sheering forces generated by a mounting event involving a bovid that can weigh well in excess of one thousand pounds (the average weight of a Holstein cow for example is about 1,500 pounds), render such devices suitable for only a single use or for detecting a single ovulation cycle.

### BRIEF SUMMARY OF INVENTION

Certain embodiments of the present disclosure are summarized below. These embodiments are not intended to limit the scope of the claimed invention, but rather serve as brief descriptions of possible forms of the invention. The invention may encompass a variety of forms which differ from these summaries.

Various embodiments of the present disclosure overcome certain deficiencies of the prior devices by providing a system having disposable components and reusable components. The disposable components are secured with the reusable components in an interlocking fashion during use. Thereafter, the components may be disengaged and a new set of disposable components can be associated with the reusable components for a subsequent use. In this way certain embodiments of the present invention provide a reusable system for detection of heat or estrus.

One embodiment provides a reusable estrus detection system having disposable components suitable for a single use and reusable components suitable for one or more uses. The disposable components can include an adhesive layer having a first side for attachment to a mammal and a second side and a housing mount secured to the second side of the adhesive layer. The reusable components can include a housing assembly releasably mounted to the housing mount and a sensor apparatus contained within the housing assembly. The sensor apparatus may include a power source coupled to a contact sensor for detecting contact and a transmitter for transmitting contact information.

In some embodiments the contact sensor and transmitter of the reusable estrus detection system are connected through a printed board circuit assembly.

In some embodiments the housing assembly includes at least one tab. In such embodiments the mounting bracket may include at least one slot that interlocks with the tab of the housing assembly to releasably secure the housing assembly to the mounting bracket.

In other embodiments the housing assembly is secured to the mounting bracket with one or more clasps, hooks, or magnets.

In some embodiments the contact sensor can be a switch, a pressure sensor, or a capacitance sensor.

In some embodiments the housing assembly is constructed to have an angled or beveled housing side. Such embodiments eliminate corners or other surfaces that may be prone to lifting.

In some embodiments a flexible material is provided in communication with the sensor apparatus. In such embodiments, the flexible material may be mounted in a window formed in the housing assembly.

In some embodiments the housing mount includes one or more clips for securing at least a portion of the assembly housing. In such embodiments the clip can include a movable element configured for securely receiving at least a portion of the cover.

In some embodiments the sensor apparatus is configured to detect mounting attempts.

In some embodiments the sensor apparatus further can include an accelerometer. In such embodiment, data generated from the accelerometer can be used to verify mounting attempts detected by the contact sensor.

In some embodiments the housing mount comprises a semi-rigid material that allows for bending or deflection. In such embodiments, the housing assembly may be dimensioned such that the housing mount is bent or deflected to secure the housing assembly to the housing mount.

In some embodiments the housing assembly can be a modular and sealed housing assembly.

Some embodiments of the present disclosure relate to a method of detecting estrus in one or more mammals. Such methods may begin by securing a reusable estrus detection system to a mammal. The reusable estrus detection system having disposable components suitable for a single use and reusable components suitable for one or more uses. The disposable components can include an adhesive layer having a first side for attachment to a mammal and a second side and a housing mount secured to the second side of the adhesive layer. The reusable components can include a housing assembly releasably mounted to the housing mount and a sensor apparatus contained within the housing assembly. The sensor apparatus may include a power source coupled to a contact sensor for detecting contact and a transmitter for transmitting contact information.

The reusable estrus detection system may then be operated to detect estrus in the mammal and then the reusable estrus detection system may be removed from the animal. Either following removal or prior to removal from the mammal the housing assembly may be released from the housing mount. Next, the disposable components may be replaced with new disposable components including a new adhesive layer having a first side for attachment to a mammal and a second side, and a new housing mount secured to the second side of the new adhesive layer. Finally, the housing assembly may be releasably mounted to the new housing mount and the reusable estrus detection system may be secured to the same mammal or a different mammal with the new adhesive layer.

In some embodiments the estrus detection device can be removed following the detection of estrus or insemination.

In some embodiments, the reusable estrus detection system is secured onto a second mammal.

In some embodiments, the one or more mammals comprise bovids.

In some embodiments the housing mount is bent or deflected in order to secure the housing assembly.

In some embodiments the one or more mammals are bovid.

Other objects and features will become apparent from the following detailed description considered in conjunction with the accompanying drawings. It is to be understood, however, that the drawings are designed as an illustration and not as a definition of the limits of the disclosure.

### BRIEF DESCRIPTION OF THE DRAWINGS

FIG. 1 depicts a view of an exemplary reusable estrus detection system mounted on a bovid in accordance with an embodiment of the invention.

FIG. 2 depicts an exploded top view of exemplary disposable components and exemplary reusable components of an embodiment of the invention.

FIG. 3 depicts an exploded top view of an exemplary housing assembly in accordance with an embodiment of the invention.

FIG. 4 depicts an exploded view of an exemplary housing assembly from below in accordance with an embodiment of the invention.

FIG. 5 depicts a side view of exemplary reusable components aligned with exemplary disposable components in accordance with an embodiment of the invention.

FIG. 6 depicts electronic components in accordance with an embodiment of the invention.

FIG. 7 depicts a view of another exemplary embodiment of a reusable estrus detection system secured to a bovid.

FIG. 8 depicts an exploded top view of disposable components and reusable components of an embodiment of the invention.

FIG. 9 depicts an exploded bottom view of disposable components and reusable components of an embodiment of the invention.

FIG. 10 depicts a block schematic representation of the electrical components in accordance with an embodiment of the invention.

### DETAILED DESCRIPTION OF THE INVENTION

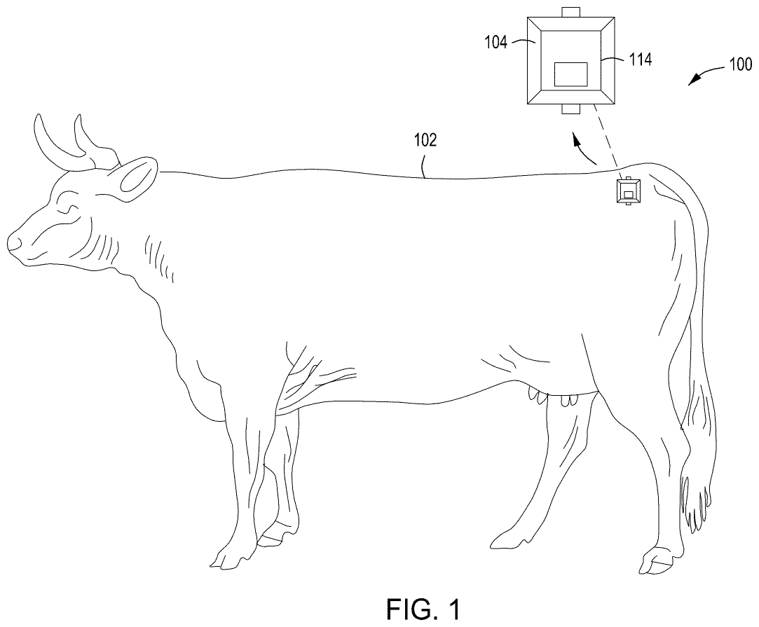

FIG. 1 illustrates an embodiment of a reusable estrus detection system 100 mounted with a mammal 102. Reusable components 114 interlock with disposable components 104 secured to a bovid. It may be appreciated, however, that the reusable estrus detection system 100 may be used with other mammalian species in which there is a desire to track mounting behaviors for the purpose of detecting estrus or timing artificial insemination. While the reusable estrus detection system 100 is placed near the tail, those of skill in the art will understand the device can be placed on other similar suitable locations for detecting mounting events.

The disposable components 104 include those components suitable for a single use, or otherwise for relatively limited use. Relative to the reusable components 114, the disposable components 104 will be low cost to manufacture and easy to replace. The disposable components 104 include those mechanical components in direct, or nearly direct, contact with the mammal as well as with a strong adhesive for securing the entire system to the mammal. The reusable components 114, on the other hand, include those electrical components, sensors and a housing that are more expensive and which have useful lifespans greater than the time required for a single estrus detection in a mammal. As will be described in greater detail in the following description, the reusable components 114 may be contained within a modular housing for easily disengaging from an interlocking relationship with a first set of disposable components and reengaging in an interlocking relationship with a second set of disposable components. In this way the reusable components can be easily reused, thus greatly improving the utility of the device and reducing the cost per use of the overall reusable estrus detection system 100.

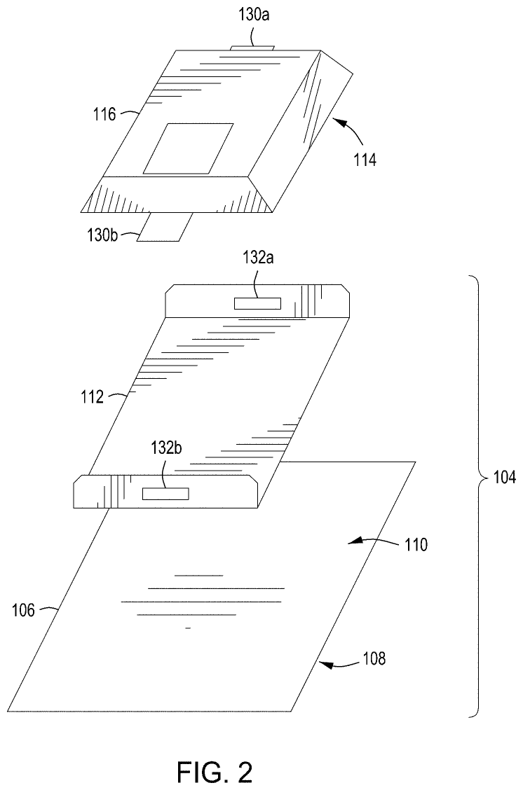

FIG. 2 depicts reusable components 114 including a housing assembly 116 having at least one tab 130 extending outwardly. The depicted embodiment includes a first tab 103a extending outward from one side of the assembly housing 116 and a second tab 130b extending outward from the opposite side of the assembly housing. Those of skill in the art can deploy different numbers and configurations of tabs in a similar manner. The disposable components 104 include an adhesive layer 106 having a first side 108 and a second side 110. The first side 108 is configured for firmly affixing the adhesive layer 106 to the mammal. A housing mount 112 is firmly affixed to the adhesive layer 106. The adhesive layer 106 may comprise a substrate having adhesive material, such as a glue, an epoxy, or another bonding agent, which may be applied just prior to application on a mammal 102. In some embodiments, the adhesive layer 106 may comprise a one sided or two-sided tape. In an embodiment utilizing two-sided tape masking material may be applied to the second side 110 of the adhesive layer 106 in order to eliminate any undesirable adhesive surface. In other embodiments the adhesive layer 106 may be contained entirely within the boundary of the housing mount 112.

The housing mount 112 may be affixed to the second side 110 of the adhesive layer 106 through adhesion or by other means. In another embodiment, the housing mount 112 may reside beneath the adhesive layer 106 and may extend through slots formed in the adhesive layer 106. The housing mount 112 depicted in FIG. 2 includes a horizontal spacing element and two vertical elements, each having a slot 132 formed thereon. The horizontal spacing element establishes a set distance between the vertical elements. This set distance corresponds to the width of the housing assembly to facilitate a force fit, or a complimentary fit between the body of the housing assembly 116 and the housing mount 112. The housing mount 112 provides a means for releasably securing the housing assembly 116 to the adhesive layer 106 and to the mammal 102. Numerous additional configurations are contemplated within the scope of broad embodiments of the invention for performing this same function. As but a few examples, the housing assembly 116 and the housing mount 112 may interlock through a force fit, complementary contact surfaces, magnets, buckles, clips, clasps or through other known means of engaging elements.

The depicted housing assembly 116 includes at least one tab 130 for mating with at least one slot 132 of the housing mount 112. A first tab 130a is positioned for interlocking with a first slot 132a and a second tab 130b is positioned to interlock with a second slot 132b of the housing mount 112. The housing assembly 116 interlocks with the housing mount 112 when the two depicted tabs 130 are inserted through each respective corresponding slot 132. Once interlocked, the housing assembly 116 is securely engaged to the housing mount 112 and the adhesive layer 106. Two tabs 130 are illustrated as relatively flat for engaging two narrow slots, but those of skill in the art can appreciate that each of the tabs and slots may have varying configurations and shapes so long as they provide an interlocking means for securing the system. As but one example, the tabs 130 and slots 132 can be dimensioned or positioned for interlocking the system in a specified orientation. In such embodiments, the dimensions of one of the tabs 130n may exceed the dimensions of all but one slot 132n, so that only the specified tab 130n can interlock with a specified slot 132n. In other alternative embodiments, the housing mount 112 could comprise at least one inward facing tab for mating with at least one slot formed in the housing assembly 116.

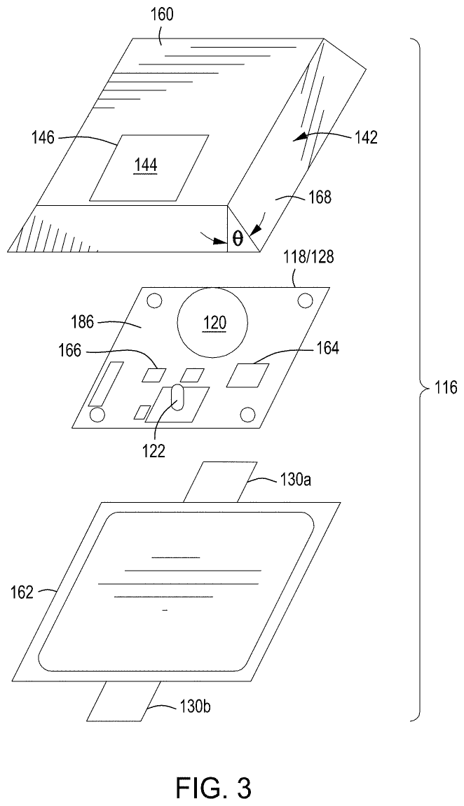

FIG. 3 illustrates an exploded top view of the housing assembly 116. A sensor apparatus 118 in the form of a printed circuit board assembly 128 resides between a housing top 160 and a housing bottom 162. A first tab 130a extends from the housing bottom 162 on a first side in a first direction and a second tab 130b extends in the opposite direction. In alternative embodiments, tabs 130, or other securing means, may be formed on the housing top 160. The depicted housing assembly 116 together with the sensor apparatus 118 contained therein serves as the functional unit for detecting and transmitting information indicative of mounting behaviors.

The printed circuit board assembly (“PCBA”) 128 consists of a printed circuit board (“PCB”) 186 assembled with additional electrical components that together perform the functions of the sensor apparatus 118. A contact sensor 122 located on the PCBA 128 provides the primary means for detecting forces representative of a mounting behavior. Exemplary contact sensors 122 can include switches, pressure sensors, capacitance sensors, hall effect sensors, and the like. The contact sensor 122 can also be a bend sensor, deflection sensor or a stain gauge properly configured with the housing assembly 116. A microcontroller 164 may store executable instructions for operation of the reusable estrus detection system 100, as well as information from the contact sensor 122. A power source 120, such as a battery, may be located on the PCBA 128 to supply power to the microcontroller 164, the contact sensor 122 as well as to other electrical components on the PCBA 128. A radio circuit 166 may transmit information for real time, or near real time, monitoring of animal mounting behaviors. In this way, information may be transmitted through a network and monitored or processed remotely by algorithms to determine whether a mammal is in estrus. Such algorithms and external monitoring programs may then proceed to designate a time or window for artificial insemination

FIG. 3 depicts angled or beveled 142 housing sides 168. As but a non-limiting example the housing sides 168 may have an angle θ between 15 to 45 degrees. The angle or bevel 142 of the housing sides 168 reduces or even eliminates edges at the top surface of the housing assembly 116 that can be caught and lifted by the mass of second mammal such as during mounting behavior or even incidental contact between large animals. The angle or bevel 142 further tends to distribute forces from such mounting behaviors and incidental contact downward and horizontally. In this way forces detrimental to the secure placement and positioning of the device may be redirected into less problematic vectors. In particular, upward and horizontal forces may have a greater tendency to overcome the adhesive resulting in the displacement or removal of an estrus detection system. The angle or bevel 142 tends to translate forces into a downward component, which is less prone to dislodge the reusable estrus detection system 100 from a mammal 102.

Those of skill in the art can appreciate other shapes of the housing assembly 116 are contemplated for reducing upward lifting forces. As but further examples, the housing assembly 116 can have rounded sides or may have an overall rounded dome shape on the top and sides.

Contact sensor 122 aligns beneath a window 146 formed in the housing top 160. The window 146 provides an opening covered with a flexible material 144. The flexible material 144 deforms under contact between mammals, such as during a mounting behavior. The contact sensor 122 not only aligns with the window 146, but also retains a depth positionally appropriate for contact with the flexible membrane 144. Alternatively, the contact sensor 122 may reside near the window 146 without touching the window 146, such that any deflection of the flexible membrane 144 triggers a contact event by the contact sensor 122. In certain embodiments, the flexible material 144 comprises a material that is transparent or semitransparent. An LED 180 may optionally be placed near the contact sensor 122 or in another location visible from the window 146. The LED 180 may indicate activation of the contact sensor 122 has been activated. The LED 180 may also indicate the system has been initialized, battery life status, or may otherwise convey information about the system to a user.

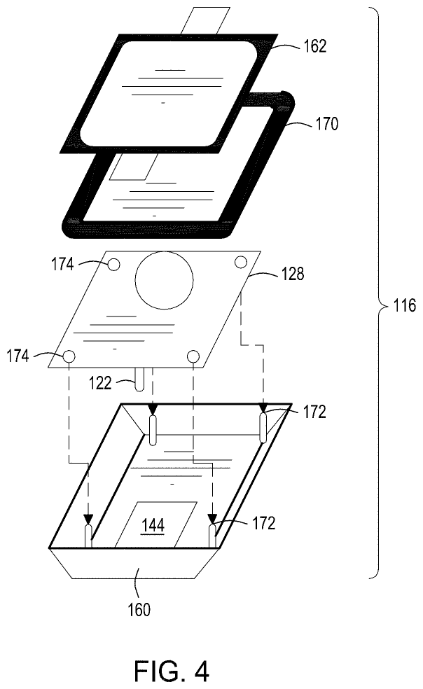

FIG. 4 illustrates an exploded bottom view of the housing assembly 116 comprising the housing bottom 162 a sealant 170 or boding agent the PCBA 128 and the housing top 160. From the depicted view four mounting elements 172 are visible in the housing top 160. The mounting elements 172 can be formed in the housing top 160, but the mounting elements 172 can also be affixed to the housing top 160 through fasteners, bonding agents, or other means. As but one example, the mounting elements 172 may comprise fasteners themselves inserted through the exterior of the housing top 160.

Mounting holes 174 formed on the PCBA 128 are spaced and dimensioned to receive the mounting elements 172 connected to the mounting top 160. In some embodiments, a plastic face may be incorporated in order to limit the upward movement of the PCBA 128 within the housing. The mounting elements 172 mate with mounting holes 174 to hold the PCBA 128 in a fixed location relative to the housing top 160. This position will, for example, align the contact sensor 122 with the flexible material 144. Those of skill in the art will appreciate numerous forms that may be molded in the housing top 160 as mounting elements 172, or which may be added by welding, fasting or other means for the purpose of enclosing a housing. The configuration of the mounting elements 172 and the mounting holes 174 may also position the PCBA 128 and the contact sensor 122 at an appropriate depth relative to the flexible material so that the contact sensor 122 is triggered when a second animal attempts to mount the first animal. In some embodiments, the contact sensor 122 may be positioned directly in contact with the flexible material 144. In other embodiments, the contact sensor 122 may be located some set distance from the flexible material 144. In such embodiments establishing a set distance, or depth, may reduce false positive signals which may be produced in response to incidental or glancing contact that is not indicative of a mounting behavior. Those of skill in the art can determine suitable distances by balancing reducing false positives caused by non-mounting contact of the flexible membrane with the desire not to miss events likely to be caused if mounting activity

The housing bottom 162 may be affixed to the housing top 160 with a sealant 170 to ensure that the housing assembly 112 remains water resistant. Alternatively, the housing bottom 162 may be bonded to the housing top 160 with a glue, a bonding agent, epoxy, or through sonic welding. In yet another embodiment, the housing top 160 and the housing bottom 162 may be secured together with fasteners. Such an embodiment may also include a gasket or seal to ensure that the housing assembly 116 remains water resistant. In each embodiment it is desirable that the electronics are protected from moisture, such as condensation, precipitation and perhaps even splashing or licking by other animals.

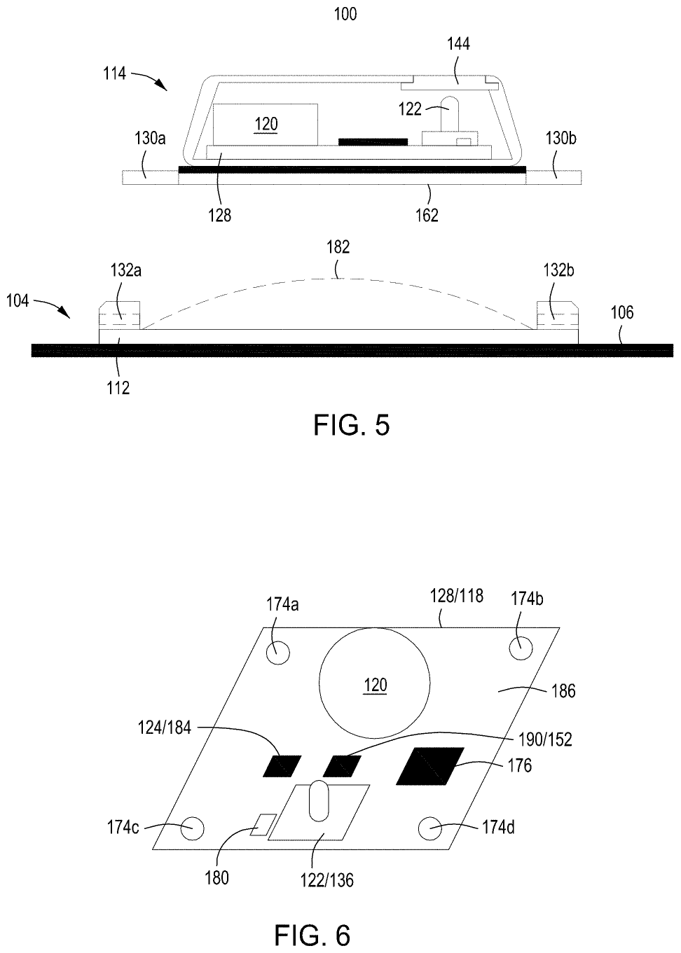

FIG. 5 depicts a side view of the reusable estrus detection system 100. The disposable components 104 are aligned with, but not interlocked with, the reusable components 114. A housing bottom 162 includes two tabs 130a,b for interlocking with the slots 132a,b of the housing mount 112. The contact sensor 122 and power source 120 are mounted to the PCB 186 in the housing assembly 116. In the depicted embodiment a small gap exists between the contact sensor 122 and the flexible material 144 so that any deformation activates the contact sensor 122. Optionally, an intermediate material may be placed between the flexible barrier and the contact sensor 122. Those of skill in the art can appreciate other sensors may be used to detect deflection of the flexible material 144. As but a few examples the contact sensor 122 can be replaced with a deflection sensor, a bend sensor, a pressure sensor, a capacitance sensor, a stain gage, a time of flight sensor, or an appropriately configured hall effect sensor.

In embodiments incorporating a hall effect sensor, the hall effect sensor may be placed in the same location as the contact sensor 122 on the PCBA 128 and the flexible material 144 could be modified with the addition of a magnet. The distance between the magnet and the hall effect sensor would be set such that any deflection of the flexible material 144 representative of a mounting behavior places the magnetic field of the magnet within range of the hall effect sensor. In other embodiments such incorporating deflection or bend sensors, those sensors may be bonded to the flexible material 144 such that deflection indicative of mounting behavior would register as a detected. Still other sensors may be readily employed to detect deflection of the flexible material 144 in configurations known to those of skill in the art.

Disposable components 104 including the adhesive layer 106 and the housing mount 112 are depicted below the reusable components 114 substantially aligned for an interlocking configuration. A deflection range 182 for the housing mount 112 indicates an exemplary deflection range for the housing mount 112. Deflecting the housing mount 112 may facilitate a two-step interlocking engagement between the housing assembly 116 and the housing mount 112. The housing mount 112 may be constructed from a semi rigid material to facilitate this deflection, such as ABS, poly propylene, or a suitable thermoform plastic.

The two-step interlocking engagement may begin with inserting first tab 130a of the housing assembly 116 into corresponding first slot 132a in the housing mount 112. Next, applying an upward force in the center of the housing mount 112 introduces a deflection 182 and the second tab 130b can be inserted into its corresponding second slot 132b. Thereafter removing the deflection 182 in the housing mount 112 causes the housing assembly 116, and specifically the housing bottom 162, to sit flush against the housing mount 112 with each tab 130 to fully engaged in each respective slot 132.

In this manner the housing mount 112 and the housing assembly 116 are securely interlocked and cannot be disengaged by a lateral motion or force, such as the forces caused by one animal sliding down off the back of the animal to which the device is affixed. An operator may remove the housing assembly 116 by reintroducing the deflection 182 into the housing mount 112 and then removing a one tab 130 from its corresponding slot 132. Once a first tab 130 has been removed, the housing assembly 116 can slip free of the remaining slot 132. Following removal of the housing assembly 116, that housing mount 112 remains affixed to the mammal 102 and can be removed later and discarded.

FIG. 6 illustrates the PCBA 128 in accordance with embodiments of the invention. The PCBA 128 serves as the sensor apparatus 118 and includes electric components mounted on a printed circuit board 186 for the purpose of generating a signal containing information indicative of mounting behaviors. The printed circuit board 186 may comprise a conductive layer and one or more insulating layers that together support and electrically connect electrical components through conductive tracks, pads and other etched features. The printed circuit board 186 can include mounting holes 174a, 174b, 174c, 174d for mounting the circuit board with the housing top 160. The power source 120 provides energy for operating the other active electrical components contained within the housing assembly 116. The power source 120 may comprise a rechargeable power source, a photovoltaic element, a chemical battery, a super capacitor, a fuel cell, a mechanical energy harvest system, or even combinations thereof.

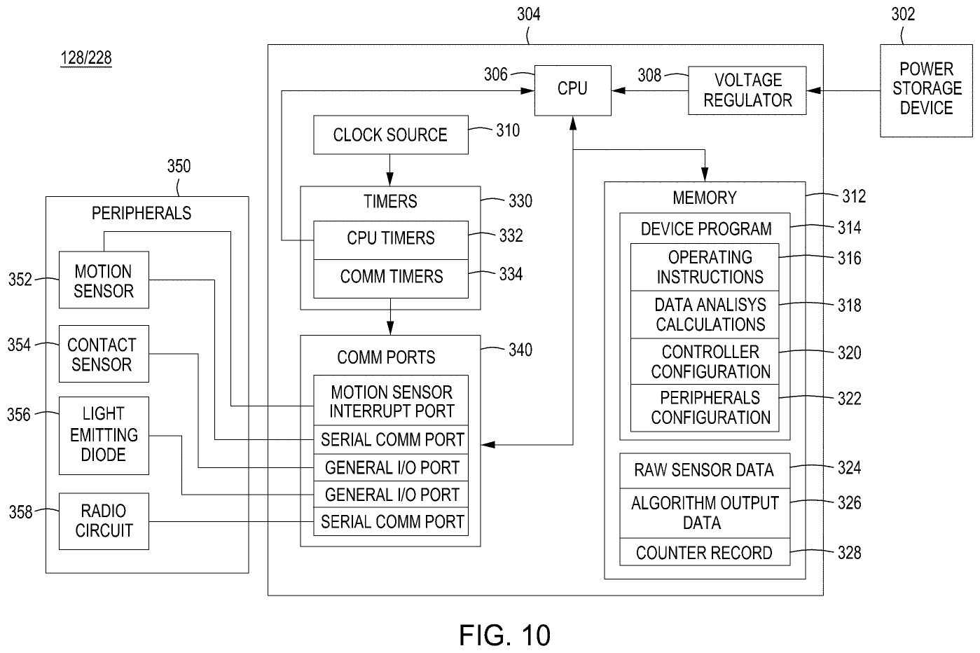

As described in more detail with respect to FIG. 10 microcontroller 176 includes a microprocessor and other integrated components for executing instructions and manipulating or storing data from various peripheral components. Exemplary peripherals contained on the printed circuit board 186 include a contact sensor 122 illustrated in the form of a switch 136, and a transmitter 124 which may be a radio circuit 184. Optionally, an LED 180 and additional sensor 190, such as a motion sensor or an accelerometer 152, may also be mounted with the PCB 186. Data from the additional sensor 190 may be evaluated along with data from the contact sensor 122 in order to determine mounting behaviors and determine the onset of estrus.

Those of skill in the art will appreciate that in some embodiments, additional sensor 190 may incorporate one or more sensor components or subsystems to determine motion, acceleration, orientation, temperature, light, sound, location including global positioning, (ruminant) pH, chemical or biochemical status, electromagnetic signal or a combination of any of these variables. Data from such various sensors can be combined and evaluated with weighting functions or other mathematical functions and algorithms to improve the accuracy and reliability of measurements.

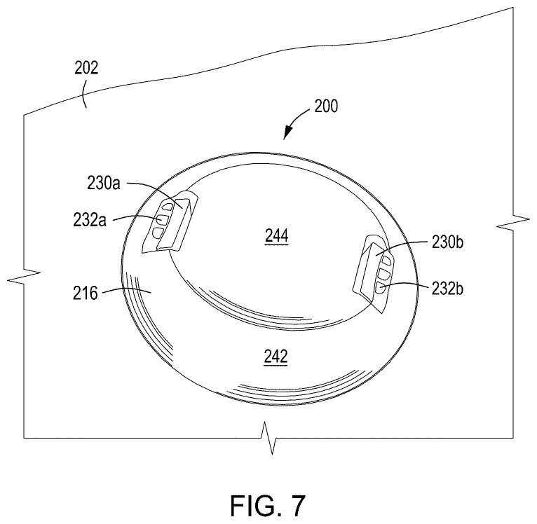

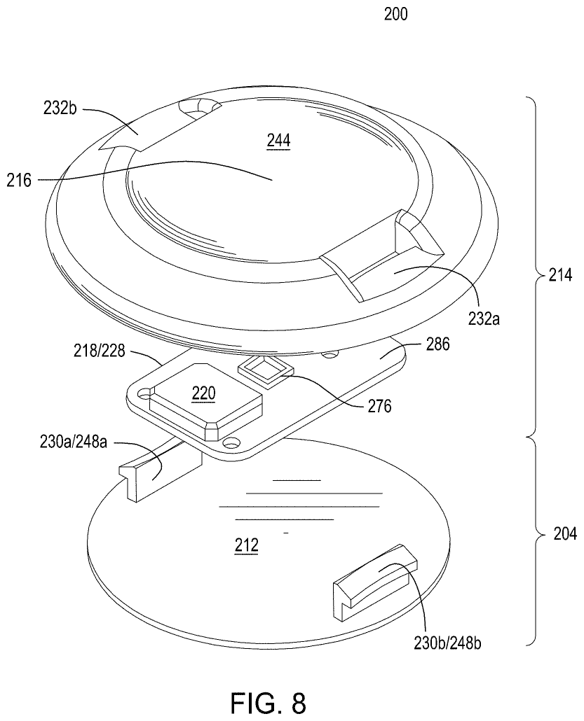

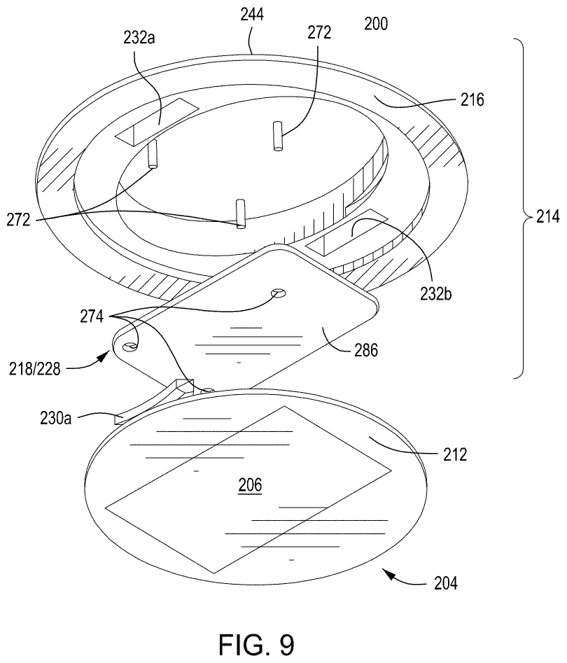

FIG. 7 illustrates another embodiment of a reusable estrus detection system 200 having reusable components 214 and disposable components 204, each better seen in FIGS. 8 and 9. Again, the disposable components 204 may comprise those low cost and easily replaceable components primarily associated with securing the reusable components 214 to a mammal, such as a bovid 202. In some embodiments the reusable estrus detection system 200 may have a generally circular footprint. Such a circular footprint may be advantageous in that vertical edges and corners are eliminated that can present contact points susceptible to snagging or otherwise being easily lifted or moved from its position on the mammal. For this reason, the round shape may better help and better secure the reusable estrus detection system 200 to a larger mammal. Similarly, the housing assembly 216 may have a beveled or curved side surface 242. The beveled or curved side surface 242 eliminates horizontal edges that could also be caught or snagged and displaced by the interaction of two large mammals, such as mounting behavior. A flexible material 244 may comprise an entire top surface of the housing assembly 216. Such an embodiment presents a greater contact surface area for detecting mounting behaviors. Further, the system 200 may be less dependent on precise location and orientation on the bovid 202 to reliably detect mounting behaviors.

As can be seen in FIG. 8, an embodiment of the reusable estrus detection system 200 includes disposable components 204 including housing mount 212 in the form of a flat disposable plate having a first tab 230a and a second tab 230b formed thereon. The housing mount 212 may be formed from a plastic, rubber, or other sufficiently rigid material for interlocking with the housing assembly 216. The reusable components 214 including the housing assembly 216 and the sensor apparatus 218 are depicted in relative alignment with respect to the disposable components 204.

In one embodiment, the housing mount 212 and the tabs 230 are constructed from a material sufficiently rigid and non-deformable to resist deformation under the weight of a bovid. At the same time, the material should be flexible enough for the tabs 230 to bend slightly as they engage with slots 232a,b formed in the housing assembly 216. In such an embodiment the tabs 230 may be formed as clips 248 for holding the housing assembly 216 firmly in place against the housing mount 212. However, other interlocking mechanisms such as clasps, hooks, magnets, fasteners or even a threaded engagement may serve a similar function. While FIG. 8 illustrates two such tabs and slots, those of skill in the art will appreciate 1, 3, 5, 6, or even more pairs of tabs and slots could also be used.

As but non-limiting examples, the exterior of the housing assembly 216 may be formed from a plastic such as polyvinyl chloride (PVC), polyethylene (PE), nylon, or acrylonitrile butadiene styrene (ABS). The flexible material 244 located at the top of the housing assembly 216 may be constructed from the same or a different material as compared to the housing assembly 216. In some embodiments, the contact point for the contact sensor will be higher than the top portion of the tabs 230, so that the tabs 230 do not interfere with the downward deflection of the flexible material 244 during a mounting activity. In another embodiment, the contact point of the contact sensor may be lower than the top portion of the tabs 230. In such an embodiment, the tabs may serve to guard the contact sensor from being triggered by glancing or incidental contact.

FIG. 8 partially reveals a sensor apparatus 218 comprising a printed circuit board assembly 228. In the PCBA 228 a power source 220 (such as a battery) and a microcontroller 276 sit on a printed circuit board 286. Other peripheral components obstructed from view include a contact sensor, a portion of which would touch or align just beneath the flexible material 244 formed in a top portion of the housing assembly 216. The contact sensor could be a mechanical switch, a pressure sensor, a capacitance sensor, a stain gage, a deflection or bend sensor, a time of flight sensor or an appropriately configured hall effect sensor.

The flexible material 244 may comprise a plastic sufficiently pliable for deformation under the weight of a mounting behavior. Without limiting the generality of the foregoing, suitable plastics may include: a plastic such as polyvinyl chloride (PVC), polyethylene (PE), nylon, or acrylonitrile butadiene styrene (ABS). Suitable materials can also include a rubber, metal, or other composite, so long as the material is capable of elastic deformation in a manner that triggers the contact sensor.

FIG. 9 depicts an exploded bottom view of the reusable estrus detection system 200 having essentially the same elements illustrated in FIGS. 7 and 8. The adhesive layer 206 is secured to the bottom of the housing assembly 212. From the bottom view the mounting holes 274 can be seen in the printed circuit board 286 for matching with the mounting elements 272 formed in the housing assembly 212.

FIG. 10 schematically represents an embodiment of the electronics that may be incorporated into the reusable estrus detection system 100/200 and which comprise the sensor apparatus 118/218 or the printed circuit board assembly 128/228. These electrical components perform three primary functions. A subset of electrical components stores and supplies power to the remainder of the active electrical components. Power supply 302 provides this function, preferably in the voltage range of 3V-3.6V, with a high maximum current draw limit. As non-limiting examples the power supply 302 (or power storage device) may comprise a coin battery such as CR2032 batteries; CR2032VP available from Energizer Battery Company and CR-2032/VCN from Panasonic-BSG. Those of skill in the art will appreciate that other power sources can also be used, including a rechargeable power source, a photovoltaic element, a chemical battery, a super capacitor, a fuel cell, a mechanical energy harvest system, or even combinations thereof.

The electronics also perform a data processing function in the system, and more particularly manage memory, timers and communications of the system. In certain embodiments, a microcontroller 304 with suitable integrated components performs these functions. Such a microcontroller 304 includes a microprocessor 306 for executing instructions stored in memory and generating various outputs. In certain embodiments the microcontroller 304 includes an integrated power regulator 308 (or voltage regulator) for managing power from the power supply 302. While an integrated power regulator 308 reduces the number of necessary additional components and reduces the complexity of the system, those of skill in the art will appreciate that alternative embodiments utilizing an external power regulator could also be used. The microcontroller 304 also includes an integrated clock source 310. Again, those of skill in the art will appreciate alternative embodiments, in which the clock source 310 is not integrated with the microcontroller 304, but rather an external oscillator in communication with the microcontroller 304. Exemplary microcontrollers 304 suitable for embodiments of the disclosed invention include part number STM32L011F3P6 available from ST Microelectronics; part number LPC1111FDH20/002.5 available from NXP USA Inc; and part number MSP430FR2311IPW20R available from Texas Instruments. Those of skill in the art will recognize that various other suitable microcontrollers may also be employed. Those of skill in the art will appreciate that microprocessors lacking certain integrated components specified above could also be utilized. Such embodiments require additional assembly and connecting additional components within the purview of those of skill in the art.

Memory element 312 includes several functional blocks of memory. A first functional block of memory characterized as device program 314 holds the operating program for the microcontroller consisting of the operating instruction code 316, data analysis calculations 318 used by the operating instruction code 316, as well as controller configuration data 320 of any multipurpose I/O ports on the microcontroller itself, and peripheral configuration setting profiles 322 for the connected peripherals. A second functional block of memory is used as storage for data collected from the various peripherals (such as raw sensor data 324), secondary data derived from the output from primary sensor data that has been through any data analysis calculations (algorithm output data 326), and the current state of any counters (counter record 328) used by either the devices operating instructions 316 or any of the data analysis calculations 318.

The integrated clock source 310 manages various timers 330 set up by the operating instructions 316. The timers 330 manage functions such as changing the microcontroller 304 awake/sleep state and activating or deactivating peripherals 350. The timers 330 may include CPU timers 332 and communication timers 334 that manage communication timing between the microprocessor 306 and the peripherals 350 to ensure data is being sent and received at that same rate.

The communication ports 340 encompass all the one way and two-way communications between the microprocessor 306 and the peripherals 350. The communication ports 340 can include general I/O lines that read or set a logic state, bi-directional serial communication ports that transfer data back and forth between the microprocessor 306 and the peripheral 350, or interrupt ports that are configured to send a signal from a peripheral 350 to the microprocessor 306 that is intended to change the microprocessor 306 from a sleep state to an awake state in order to catch an unpredictable event. In one embodiment of the reusable estrus detection system 100/200, there are four exemplary peripherals 350; a motion sensor 352, a contact sensor 354, a light emitting diode 356, and a transmitter such as a radio circuit 358. Each such peripheral will be in communication with an appropriate communications port 340.

Those of skill in the art will appreciate the peripherals 350 specified in FIG. 10 represent an exemplary subset of peripherals 350 that may be incorporated in embodiments of the invention. Certain embodiments may minimally include the contact sensor 354 and a transmitter 358. The transmitter 358 may comprise a radio circuit or any other device capable of transmitting encoded data using a specific frequency range in the electromagnetic spectrum. Those of skill in the art would be aware of suitable transmitters and frequency ranges for the embodiments described herein. Broadly speaking, the transmitters may operate in normal radio frequencies ranges, they may also communicate by blue tooth, cellular, or wi-fi. As specific examples of radio circuits, the following radio circuits may be employed: part number CC1101RGPR available from Texas Instruments; part number AX5043-1-TW30 available from ON Semiconductor; part number MRF89XAT-I/MQ available from Microchip Technology.

In certain embodiments of the invention the contact sensor 354 can be a momentary switch, a pressure switch, a pressure sensor, a capacitive contact sensor, an inductive contact sensor, a time of flight sensor or other known sensors that can be configured to measure contact of the deflection of a material, such as bend sensors or even hall effect sensors. Any other sensors suitable to detect the proximity of a second mammal or a pressure or deflection of a portion of the reusable estrus detection system may also be used. Specific switches that may be incorporated into embodiments of the present invention include part number FSM4JSMATR available from TE Connectivity ALCOSWITCH Switches; part number FSM6JSMATR available from TE Connectivity ALCOSWITCH Switches; and part number EVQ-Q1E06K available from Panasonic Electronic Components.

Motion sensor 352 could include any number of devices ranging from a tilt sensor, to an accelerometer, to a gyroscope. Such sensors could be used to detect changes in orientation of the reusable estrus detection system 100/200 and interface with the microcontroller 304. Exemplary motion sensors that may be incorporated into embodiments include part number ADXL343BCCZ-RL from Analog Devices Inc.; part number LIS2DH12TR available from ST Microelectronics; and part number FXLS8471QR1available from NXP USA Inc.

A light emitting diode (LED) 356 can provide visual feedback to a user about conditions of the reusable estrus detection system 100/200 such as positive confirmation of commissioning or decommissioning of the device, activation or deactivation of a peripheral 350, or an indication sensed data or mathematically processed data meet a threshold or requirement. The LED 356 itself could be replaced with any kind of visual notification element and that performs a similar function. As one of skill in the art can appreciate the operating instructions 316 may provide for numerous illumination patterns to communicate any or all of the conditions specified above.

Those of skill in the art can readily appreciate additional peripherals in the form of additional sensors, such as motion sensors, accelerometers, and gyroscopes which may provide information for determining mounting behaviors as well as additional sensors that record position and temperature such as a GPS and a thermometer. Additional components may also be employed on the PCB such receivers and transceivers for wireless communication such as by wi-fi, blue tooth or the like. In some embodiments, a voltage regulator and oscillating timing circuit are disposed on the PCB rather than integrated with the microcontroller.

An exemplary method in accordance with embodiments of the claimed invention may begin with securing a reusable estrus detection system having disposable components and reusable components to a mammal, such as a first bovid. The reusable estrus detection system may be like those disclosed in embodiments of the system described above or may include alternate or optional features described herein so long as the system is generally characterized by having both a reusable portion and a disposable portion.

Securing the system to a mammal may further include the step of binding an adhesive layer to one or more other disposable components. The disposable components, such as a housing mount, may then be secured to the bovid in a suitable placement for estrus detection. For example, the system may be mounted near the hind portion or near the tail of the mammal. In such embodiments the adhesive layer may comprise a two-sided adhesive tape, such as available from 3M. Alternatively, an adhesive or bonding agent may be applied to the adhesive layer just prior to its application onto the animal.

The reusable components may be interlocked with the disposable components such that the entire system is secured to the mammal through the adhesive layer. Importantly, the step of interlocking the disposable and reusable components may occur before or after the disposable components are mounted to the animal with adhesive. In configurations previously described in detail, tabs or clasps from either the housing mount or the housing assembly may be paired with the corresponding slots in the other. Alternatively, the housing mount and the housing assembly may be interlocked through other simple means, such as hooks, threading or other force fit interlocking components. The housing assembly and the housing mount may also be interlocked through interlocking pieces, such as spring-loaded interlocking mechanisms, magnets or the like.

Once secured to the mammal, the reusable estrus detection system is operated to detect estrus in the mammal. In operation, the system may detect each mounting attempt or collective mounting behaviors and relay such information to another location through network connections, wi-fi, cellular, or other radio communication means. Information may be compiled from multiple systems at a central location and may be filtered or otherwise further processed through one or more algorithms in an effort to more accurately and precisely determine the onset of estrus in bovid.

Algorithms may be stored in a memory block of the microcontroller for making determinations regarding estrus within the system. Alternatively, raw data or filtered data can be transmitted from the system for further processing at an external location in order to make the determination an animal is in estrus. Once estrus is detected, alerts can be generated, or an insemination schedule can be compiled for the producer.

A use, which may be the first use or a subsequent use, can be complete once estrus is detected, when the animal is subsequently scheduled for artificial insemination, when the animal dies, or when the animal is otherwise removed from a breeding program. Once such a use of the system is complete, the housing assembly is removed from the animal and from the remaining disposable components of the system. Importantly, the step of removing the housing assembly from the animal and the disposable components from the animal does not confer any specified order. The system may be removed at the adhesive layer from the animal followed by disengaging the interlocking reusable components from disposable components. Equally, however, the housing assembly can be disengaged from the housing mount while the housing mount is affixed to the mammal through the adhesive layer.

In certain embodiments, the housing assembly may be disengaged from its interlocked relationship with the housing mount by disengaging the tabs from the slots, either one at a time, or at the same time. In other embodiments specific motions or applications of force, such as rotating, twisting, bending or the like may be imparted upon one or both of the housing assembly and the housing mount for the purpose of disengaging their interlocking relationship.

Once removed from the animal and disengaged from the reusable components, the disposable components may be discarded, recycled, or otherwise disposed of in an appropriate manner for their specific construction materials. A second set of disposable components may then be associated with the reusable components for a second use of the reusable estrus detection system 100/200. The second set of disposable components may comprise an identical or nearly identical set of components including a second housing mount for interlocking with the housing assembly and a second adhesive layer for adhering the system to a second animal.

The reusable estrus detection system may then be secured to a mammal for a subsequent use by binding the second adhesive layer to the mammal. In some embodiments, the same animal may be the subject of both the previous and the subsequent uses, while in other embodiments the subsequent use may be on a different animal. In either event, the disposable components, such as a housing mount, may then be secured to the bovid in a suitable placement for estrus detection. The new disposable components may include a new adhesive layer in the form of a two-sided adhesive tape, such as available from 3M. Alternatively, an adhesive, epoxy or other bonding agent may be applied to the adhesive layer just prior to its application onto the animal.

The reusable components may be interlocked with the disposable components for the subsequent use in the same manner as described above with respect to the previous use. Again, the step of interlocking the disposable and reusable components may occur before or after the disposable components are mounted to the animal with adhesive.

Those of skill in the art will appreciate the exemplary method may be repeated more than once and in fact may be repeated any number of times within the spirit and scope of the claimed invention. A practical limitation on the number of uses for the reusable components may reside in the battery life. Those of skill in the art will appreciate that rechargeable batteries may be used in order to prolong battery life. Alternatively, the reusable components may be refurbished after some number of uses. As but one example the microcontroller may contain instructions for operating an LED in a specified manner when battery life reaches a certain threshold. Following use, or even mid-use, when the LED indicates low battery life a producer may remove the housing assembly and replace the battery. Use of the disposable components may then resume or a new use may even begin following battery replacement depending on the stability and status of the disposable components.

The particular embodiments or elements of the invention disclosed by the description or shown in the figures or tables accompanying this application are not intended to be limiting, but rather illustrative of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “transmitter” should be understood to encompass disclosure of the act of “transmitting”—whether explicitly discussed or not—and, conversely, were there effectively disclosure of the act of “transmitting”, such a disclosure should be understood to encompass disclosure of a “transmitter” and even a “means for transmitting.” Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

Moreover, for the purposes of the present invention, the term “a” or “an” entity refers to one or more of that entity. As such, the terms “a” or “an”, “one or more” and “at least one” can be used interchangeably herein. All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment.

The background section of this patent application provides a statement of the field of endeavor to which the invention pertains. It is not intended that any United States patent, patent application, publication, statement or other information cited or incorporated herein be interpreted, construed or deemed to be admitted as prior art with respect to the invention.

The claims set forth in this specification, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

1. A reusable estrus detection system comprising:
disposable components comprising: i) an adhesive layer having a first side for attachment to a mammal and a second side, and ii) a housing mount secured to the second side of the adhesive layer; and
reusable components suitable for one or more uses, the reusable components comprising: i) a housing assembly configured for releasably interlocking with the housing mount, ii) and a sensor apparatus contained within the housing assembly, the sensor apparatus having a contact sensor for detecting contact and a transmitter for transmitting contact information.

2. The reusable estrus detection system of claim 1, wherein the contact sensor, and transmitter are connected through a printed board circuit assembly.

3. The reusable estrus detection system of claim 1, wherein the housing mount further comprises at least one tab.

4. The reusable estrus detection system of claim 3, wherein the housing assembly further comprises at least one slot, and wherein the at least one slot of the housing assembly mates with the at least one tab of the housing mount to releasably secure the housing assembly to the housing mount.

5. The reusable estrus detection system of claim 1, wherein the housing assembly further comprises at least one tab.

6. The reusable estrus detection system of claim 5, wherein the housing mount further comprises at least one slot, and wherein the at least one slot of the housing mount mates with the at least one tab of the housing assembly to releasably secure the housing assembly to the housing mount.

7. The reusable estrus detection system of claim 1, wherein the housing assembly is secured to the housing mount with one or more clasps, hooks, or magnets.

8. The reusable estrus detection system of claim 1, wherein the contact sensor comprises a switch, a pressure sensor, or a capacitance sensor.

9. The reusable estrus detection system of claim 1, wherein the housing assembly comprises an angled or beveled housing side.

10. The reusable estrus detection system of claim 1, wherein the housing assembly comprises a flexible material in communication with the sensor apparatus.

11. The reusable estrus detection system of claim 10, wherein the flexible material comprises a top plastic layer of the housing assembly.

12. The reusable estrus detection system of claim 10, wherein the flexible material is mounted in a window formed in the housing assembly.

13. The reusable estrus detection system of claim 1, wherein the housing mount comprises at least one clip for securing at least a portion of the assembly housing.

14. The reusable estrus detection system of claim 13, wherein the at least one clip includes a movable element configured for securely receiving at least a portion of the cover.

15. The reusable estrus detection system of claim 1, wherein the sensor apparatus is configured to detect mounting attempts.

16. The reusable estrus detection system of claim 1, wherein the sensor apparatus further comprises an accelerometer.

17. The reusable estrus detection system of claim 16, wherein data generated from the accelerometer is used to verify mounting attempts.

18. The reusable estrus detection system of claim 1, wherein the housing mount comprises a semi-rigid material that allows for bending or deflection.

19. The reusable estrus detection system of claim 18, wherein the housing assembly is dimensioned such that the housing mount is bent or deflected to secure the housing assembly to the housing mount.

20. The reusable estrus detection system of claim 1, wherein the housing assembly comprises a modular and sealed housing assembly.

21. A method of detecting estrus in one or more mammals comprising:
securing a reusable estrus detection system to a mammal, the reusable estrus detection system comprising:
disposable components suitable for a single use, the disposable components comprising: i) an adhesive layer having a first side for attachment to a mammal and a second side, and ii) a housing mount secured to the second side of the adhesive layer; and
reusable components suitable for one or more uses, the reusable components comprising: i) a housing assembly releasably mounted to the housing mount, ii) and a sensor apparatus contained within the housing assembly, the sensor apparatus having a power source coupled to a contact sensor for detecting contact and a transmitter for transmitting contact information;
operating the reusable estrus detection system to detect estrus in the mammal;
removing the reusable estrus detection system from the mammal and releasing the housing assembly from the housing mount;
replacing the disposable components with new disposable components including i) a new adhesive layer having a first side for attachment to a mammal and a second side, and ii) a new housing mount secured to the second side of the new adhesive layer; and
securing the reusable estrus detection system to the same mammal or to a different mammal with the new adhesive layer.

22. The method of claim 21, wherein the estrus detection device is removed following the detection of estrus or insemination.

23. The method of claim 21, wherein the step of securing the reusable estrus detection system to the same mammal or to a different mammal comprises securing the estrus detection device onto a second mammal.

24. The method of claim 21, wherein the one or more mammals comprise bovids.

25. The method of claim 21, wherein the housing mount is bent or deflected in order to secure the housing assembly.
